(19) United States
(12) Patent Application Publication
(10) Pub. No.: US 2006/0193781 A1
(43) Pub. Date: Aug. 31, 2006

(54) Magnetic resonance imaging of metal concentrations

(76) Inventors: Christopher Frederickson, Galveston, TX (US); Paul M. Henrichs, Houston, TX (US)

Correspondence Address: Adler & Associates, 8011 Candle Lane, Houston, TX, 77071, US

(21) Appl. No.: 10/516,754

(22) PCT Filed: May 30, 2003
(86) PCT No.: PCT/US03/16935

§ 371 Date: Feb. 8, 2006

(30) Foreign Application Priority Data
Jun. 3, 2002 (US) 60384721

(51) Int. Cl. A61K 49/10; A61B 5/055

(52) U.S. Cl. 424/9.36; 600/412

(57) ABSTRACT

Provided herein is a method of magnetic resonance imaging (MRI) for in vivo mapping of concentration of a target metal ion in at least one tissue using a contrast agent selectively sensitive for the amount of target metal ion where the contrast agent itself contains a non-hydrogen imaging nucleus. Also provided a method of diagnosing a disease state and of monitoring the efficacy of a therapeutic regimen to treat the disease state using the magnetic resonance imaging methods.

### BACKGROUND OF THE INVENTION

1. Field of the Invention

The present invention relates generally to the fields of magnetic resonance imaging and diagnostic medicine. More specifically, the present invention relates to the use of contrast agents with magnetic resonance imaging (MRI) for the purpose of detecting variations in the concentration of metal ions in tissue.

2. Description of the Related Art

Magnetic resonance imaging, as it generally is practiced, is a diagnostic and research procedure that uses high magnetic fields and radio frequency signals to generate signals from which an image can be obtained. The signals result from the interaction of certain atomic nuclei with a magnetic moment with particular radio frequencies in the presence of a magnetic field.

The most abundant molecular species in biological tissues is water. Typically, the quantum mechanical “spin” of the hydrogen nuclei in the water is the source of the signals that give rise to a magnetic resonance image. In special cases, the nuclei of other atoms, such as phosphorus or fluorine, can be used for standard magnetic resonance imaging. Magnetic resonance images are typically displayed on a gray scale with black indicating the lowest and white the highest measured intensity. However, many other forms of representation are possible.

In the absence of the physical phenomenon of nuclear relaxation, which takes place at different rates for nuclear spins in different chemical and physical environments, the measured intensity for each pixel in a magnetic resonance image would be proportional to the number of spins in the spatial region represented by the pixel. However, when hydrogen is the imaging nucleus, the signals from which the image is generated come largely from hydrogens in water molecules in the body, and, as the water concentration in the soft tissues of the body is remarkably uniform, the intensity differences actually observed are much greater than can be explained by concentration variations alone. In fact, the intensity of the observed signals for each pixel is strongly influenced as to how the nuclei respond to external perturbations through the processes of longitudinal and transverse relaxation.

For a sample in an applied external field, the longitudinal component of magnetization is that component of induced magnetization directed in the same direction or against the direction of the applied magnetic field. The transverse magnetization is that component of magnetization perpendicular to the applied field. The longitudinal and transverse relaxation times T₁ and T₂ measure the rate at which the longitudinal and transverse nuclear magnetizations approach equilibrium after a perturbation of the nuclei through radio frequency pulsing or a change of conditions. For a collection of nuclei with a magnetic moment at equilibrium in an applied magnetic field, the longitudinal magnetization is non-zero and has a magnitude determined by the gyromagnetic ratio of the nucleus and the magnitude of the applied magnitude field. The transverse component has an equilibrium magnitude of zero.

Application of suitable radio-frequency pulses can disturb the longitudinal magnetization and create a non-zero transverse magnetization. The longitudinal magnetization for each nuclear species in a particular chemical environment returns towards the equilibrium value during a period comparable to the longitudinal relaxation time. The transverse magnetization precesses about the applied magnetic field at the observation frequency, i.e., the Larmor frequency, and decays towards zero over a period comparable to the transverse relaxation time. When an appropriate pick-up coil surrounds the sample, the precessing transverse magnetization induces an observable signal in the coil. It is this signal that is used for generation of a magnetic resonance image.

Stochastic processes that modulate the magnetic environment of a nucleus cause both longitudinal and transverse relaxation. Frequency components of the stochastic process at one and two times the Larmor frequency cause longitudinal relaxation. Frequencies close to zero, as well as at one and two times the Larmor frequency, cause transverse relaxation.

A major relaxation source for many nuclei is modulation of the dipolar nuclear interactions among like or unlike nuclei by molecular tumbling. For small, rapidly tumbling molecules, a decrease in the tumbling rate results in a reduction of both T₁ and T₂ of the nuclei contained by the molecule. Thus, an increase in the mass of a molecule through complexation with a metal ion would be expected to result in a decrease in the relaxation times. Modulation of electron-nuclear interactions by molecular tumbling is important when a nucleus is contained in a paramagnetic molecule. Complexation with a paramagnetic metal ion should decrease both the longitudinal and transverse relaxation times of the nuclei in a molecular complexing agent. Modulation of the nuclear-quadrupole interaction by molecular tumbling is important when the nucleus has a quadrupolar moment. In these cases, the nuclear relaxation times can be affected by the rate of electronic or quadrupolar relaxation as well as by molecular tumbling.

Some especially slow processes, i.e., those with negligible high-frequency components, can cause transverse relaxation while having little effect on longitudinal relaxation. Among the more typical of such processes is chemical exchange, in which a dynamic process leads to modulation of the chemical environment of the observed nucleus. Typically, such processes involve interchange of two different chemical species. When such interchange takes place especially slowly, separate resonance signals for the different chemical species can be observed and separate transverse relaxation times can be defined for the nuclear species responsible for each observable signal. In the absence of other sources of transverse relaxation, the inverse transverse relaxation time, 1/T₂, for each species would be equal to the rate constant for conversion of the chemical structure containing the nuclear species into another chemical structure.

Experimental procedures to exploit the differences in the relaxation properties of nuclei located in different regions of a human or non-human body for generation of a magnetic resonance image are well developed. In fact, they are an essential component of the experimental procedure for creation of such an image. A typical magnetic resonance imaging scan of a human or animal body involves application of a series of radio frequency pulses and magnetic field gradients followed by data acquisition. Multiple repetition of the process, combined with signal averaging of all the measured scans, provides signal enhancement.

The signal amplitude recorded for any given scan for each pixel is related to the extent to which the magnetization associated with the pixel has returned to equilibrium since the previous scan, as well as to the number of nuclei giving rise to the observable signal from the pixel. As indicated, the rate of recovery of the longitudinal magnetization following a perturbing pulse is measured by the longitudinal relaxation time T₁. After multiple scans, the signal intensity is suppressed for those pixels in which the longitudinal relaxation time is long compared with the time between scans. Those nuclei having the shortest longitudinal relaxation times give the largest signals.

The widespread use of spin-echo sequences for generation of the signals used in magnetic resonance imaging allows further modification of the signal intensity of each pixel. Such a sequence involves the application of multiple pulses and delayed signal acquisition. During the delay, the signal of each pixel decays at a rate determined by its transverse relaxation time T₂. When the delay is short compared with the transverse relaxation time of the observed nucleus, the observed signal intensity is suppressed. When the observed nuclei giving rise to the signals for different pixels have different relaxation times, the signal intensity for all pixels is degraded, but the intensity for those pixels associated with nuclei having short transverse relaxation times are especially degraded.

Zinc is an essential biological ion. Too little zinc is fatal, both for individual cells, when harsh intracellular chelating agents strip out the available zinc, and for intact animals, when the diet provides insufficient zinc. For reasons that are still poorly understood, the concentration of Zn⁺² in seminal fluid is especially high, reportedly around 2 mM (1-3). Nonetheless, too much zinc is also lethal. It has been shown, in several laboratories and in several different animal models, that whenever the free zinc ion concentration inside cells exceeds a few micromolar, the cells generally degenerate through apoptotic and/or necrotic injury.

As a result of the crucial biological role of zinc, the ability to detect abnormal zinc concentrations in tissues has the potential to be an indicator for certain disease conditions. For example, early detection of high zinc concentrations in the brain could be a warning of the development of Alzheimer's disease. The zinc concentration in the plaques and tangles characteristic of Alzheimer's disease can be as high as 1 mM (4). Monitoring tissue zinc concentration may also be useful in therapy. Bush et al. have shown that amyloid plaques can be resolubilized by the use of a zinc chelator. Thus, the possibility of developing drugs to treat Alzheimer's disease through the modification of the zinc levels in the brains exists. The ability to follow the course of such treatments will be useful.

Interestingly, zinc released during head injuries, seizures, or transient ischemic attacks may accelerate the pathology of Alzheimer's disease. The primary source of the released zinc, which can kill or injure neurons, is the sequestered zinc in the presynaptic vesicles of axonal boutons. Stored in concentrations of up to 1 mM in vesicles, this zinc can be released in a sudden, precipitous “flood,” from the presynaptic boutons during ischemic, traumatic, or paroxysmal events. During such episodic “floods” of Zn, the released zinc is likely to induce “growth spurts” in both plaques and tangles. Indeed, there is evidence that seizures, trauma, and ischemia do induce modifications of amyloid and APP metabolism consistent with accelerated plaque formation (5-7).

There is a potential for the development of drugs to be used as neuroprotectants in the immediate aftermath of stroke, cardiac arrest, convulsions, or traumatic head injury. In these cases, a zinc buffer(s) could be administered at the earliest opportunity, on site, by paramedics. One critical issue with regard to treatment with zinc is, of course, that the chelation of intracellular zinc can be harmful or even fatal to cells (8). Koh et al. were able to reduce neuron death by nearly one half by chelating zinc after ischemia (9). Several groups have confirmed that selective chelating of zinc in such a way that the concentration of Ca⁺² or Mg⁺² are left unaffected is a potent neuroprotective treatment (10-13).

A convenient means of showing the development of abnormally high or low concentrations of tissue zinc or for monitoring the course of therapy would be with an imaging map, in which the intensity of each pixel reflects the zinc concentration at that site. In such a map, contrast would reflect the variations in zinc concentration across the tissues being examined. Similar maps for other metal ions would be useful tools relating to other types of disease conditions.

Such maps can be generated for excised tissue slices examined under a microscope through the use of fluorescent dyes whose fluorescence is quenched upon complexation of the molecule with a metal. However these methods are affected by photobleaching of the fluorescent dye and light scattering. Additionally, such methods are not amenable to imaging of intact organisms.

It is readily apparent that the human body is almost impenetrable by visible light. Radiation in the near-infrared wavelength range penetrates tissue much more readily than does radiation in the visible range. However, strong light scattering makes generation of an image by illuminating the body with near-infrared radiation and detection of the radiation passing through the body extremely difficult. It is unlikely that high-resolution images generated with near-infrared radiation alone will ever be achieved. Generation of a suitable contrast agent whose fluorescence properties are affected by selective complexation with a target metal ion presents an additional challenge.

An alternative approach to mapping the concentrations of metal ions in the body that do not involve optical methods is required. Currently, the available methods of generating a magnetic resonance image that reflects zinc concentration have been severely limited. Magnetic resonance imaging with irradiation and detection at the ⁶⁷Zn frequency is theoretically possible. Then the image intensity at each pixel would directly reflect the zinc content of that pixel. In practice, this approach would be very difficult, if not impossible. Although ⁶⁷Zn has spin 5/2 and is suitable, in principle, for nuclear magnetic resonance, its isotopic abundance is only 4.1%. Furthermore, its resonance frequency is only 0.0673 that of ¹H. The relative sensitivity in comparison with ¹H is 2.85×10⁻³. Consequently, the signal strength is inherently very low. It is unlikely that a signal suitable for the generation of a magnetic resonance image will ever be observable for the zinc in tissue above the level of the noise. An indirect approach to measurement of zinc concentrations and, probably other metal ions, involving detection of the concentration through observation of a sensitive nucleus such as ¹H or ¹⁹F is required.

The method taught by Meade et al. is one indirect method applicable for detection of zinc and other target metal ions. It involves proton magnetic resonance and the use of blocked paramagnetic contrast agents. Most contrast agents contain paramagnetic ions, especially gadolinium ions. When water complexes with the gadolinium, the water protons relax very rapidly. Because a group of water molecules interchange with whatever water molecule is bound to the gadolinium ion, a single gadolinium has the effect of shortening the relaxation times of a large collection of water protons. Thus, relatively small concentrations of gadolinium compounds can have a large effect on the relaxation times of the water in which the compound is dissolved.

Safety prohibits the simple administration of free gadolinium ions to human patients and the art of creating contrast agents for magnetic resonance imaging involves the creation of complexation agents that sequester the gadolinium ions and reduce the toxicity of the metal to tolerable levels. In the design of contrast agents for MRI normal practice is to leave at least one complexation site of the metal ion free for interaction with water. Meade et al. teach that it is possible to construct a nascent contrast agent in which water access to any site on the paramagnetic ion is blocked by a removable functional group. Without free access of the water molecules to the inner coordination sphere of the paramagnetic ion the nascent contrast agent is relatively ineffective in shortening the longitudinal relaxation times of the water protons. Activation takes place when the blocking group is removed, either completely or partially, by interaction of the nascent contrast agent with an enzyme or metal ion. The ability of the contrast agent to relax water protons is then maximized.

The blocking agent might simply be a cap attached to the chelating group by linking groups that are subject to enzymatic cleavage. In the presence of an appropriate enzyme the blocking group is either completely removed or partially broken from the nascent contrast agent so that it no longer blocks water access. This approach may, or may not, be suitable for metal detection. In an alternative form of the nascent contrast agent the blocking group can be attached directly to the gadolinium ion. When the nascent contrast agent contacts a metal ion that interacts with the blocking group more strongly than does the gadolinium ion, the blocking group is removed. The gadolinium binding site then becomes accessible to water molecules. In either case, the interaction of the nascent contrast agent with the target species converts it from a relatively weak contrast agent into a strong contrast agent. Where the concentration of the target species is high, the longitudinal relaxation times of the water protons are shortened and the signal is enhanced.

Obviously, the design of the blocking species in this approach requires considerable chemical expertise. Furthermore, various processes may lessen the effectiveness of the agent. For example, diffusion of the contrast agent from the site where it is active could lead to relaxation of water protons far removed from the site of the target enzymes or metals.

Hanaoka et al. (16) utilize a Zn²⁺-sensitive contrast agent containing a chelated paramagnetic gadolinium (Gd³⁺) ion whose access to water molecules is modulated by the presence or absence of Zn²⁺. The method utilizes a nuclear magnetic resonance signal based on the protons in water where the T₁ and T₂ relaxation times are shortened by rapid relaxation of protons in the inner-sphere molecules complexed to the gadolinium ions accompanied by rapid exchange of inner-sphere water molecules with bulk water. Again this requires sequestering the gadolinium ions thereby reducing the toxicity of the metal to tolerable levels.

The complexing agent 1,2-bis-(2-amino-phenoxy)ethane-N,N,N′,N′-tetraacetic acid (BAPTA) binds both calcium and zinc ions. However, the dissociation constant of the zinc complex is about two orders of magnitude smaller than that of the calcium ion. When present in sufficient concentration in intracellular fluid, 1,2-bis-(2-amino-phenoxy)ethane-N,N,N′,N′-tetraacetic acid will form complexes almost exclusively with zinc ions.

The compound 1,2-bis-(2-amino-phenoxy)ethane-N,N,N′,N′-tetraacetic acid itself has the potential to give hydrogen, carbon, nitrogen, and oxygen magnetic resonance signals. Of these, only the hydrogen signal is intense. However, even the relatively strong hydrogen signal must still compete in the body with the much stronger signal of the abundant water protons. It would be very difficult, if not impossible, to separate the proton signal of 1,2-bis-(2-amino-phenoxy)ethane-N,N,N′,N′-tetraacetic acid, or a derivative thereof, that had been administered to a human or non-human patient from the background signal from the body itself. Seeing the effect of metal complexation on the magnetic resonance signal would be still further difficult. Likewise, it would be difficult to detect the proton signal of any other chemical compounds that bind strongly with zinc or any other of the metal ions found in high abundance in the body.

Fluorinated derivatives of 1,2-bis-(2-amino-phenoxy)ethane-N,N,N′,N′-tetraacetic acid, such as 5F-BAPTA and 5T-BAPTA have similar chemical properties to BAPTA and give fluorine magnetic resonance signals. ¹⁹F is a spin nucleus with near 100% natural abundance. The sensitivity relative to ¹H is 0.83 and the NMR frequency at 400 MHz is 376.31. The natural concentration of fluorine in the body is very low. Therefore, the background signal, against which the fluorine signal of a fluorine-containing compound that has been administered to the body is measured, is very low. Detection of the fluorine magnetic resonance signal of a fluorinated compound introduced into the body is relatively simple.

Metal exchange between the Zn²⁺ and Ca²⁺ complexes of both 5F-BAPTA and 5T-BAPTA occurs too slowly to average the fluorine resonances of the zinc or calcium complexes and the free 5F-BAPTA. Likewise, chemical exchange between the metal complexes and the free complexing agents is slow. As a result, when 5F-BAPTA is present in a living cell, the two complexes and the free 5F-BAPTA give separate signals. The fluorine chemical shifts of the metal complexes of both 5F-BAPTA and 5T-BAPTA differ significantly from those of the free complexing agents. For example, the fluorine signal of the 5F-BAPTA/Ca²⁺ complex is about 4.7 ppm to higher frequency than that of the 5F-BAPTA; the signal of the 5F-BAPTA/Zn⁺⁺ complex is about 3.7 ppm to higher frequency (14).

In the absence of incomplete relaxation of the nuclear spins between scans (saturation), the area under the peak of a magnetic resonance signal is determined by the number of nuclei giving rise to the signal. It has been demonstrated that 5F-BAPTA can be used to measure intracellular calcium concentrations in vivo (15). These results suggest that fluorine magnetic resonance imaging could be used to generate images reflecting tissue zinc concentrations. Experimental procedures to generate magnetic resonance images with an “extra” dimension to reflect chemical shift are available. In theory, these methods could be used to generate a map of zinc concentrations through a fluorinated 1,2-bis-(2-amino-phenoxy)ethane-N,N,N′,N′-tetraacetic acid derivative.

The initial step would be perfusion of the tissue of interest with the fluorinated 1,2-bis-(2-amino-phenoxy)ethane-N,N,N′,N′-tetraacetic acid derivative, either by intravascular injection or direct injection into the tissue. An image with the “extra” dimension sensitive to chemical shift would then be generated. Separate images reflecting either the zinc concentration or the calcium concentration would be picked out of the full imaging by selection of the image components associated with either chemical shift.

In practice, generation of a zinc image based on the chemical shift differences between free and bound forms of a fluorinated derivative of 1,2-bis-(2-amino-phenoxy)ethane-N,N,N′,N′-tetraacetic acid would be very difficult. The problem is obvious to those skilled in the art of magnetic resonance imaging. Each new dimension that is added to an image increases exponentially the amount of data that must be collected. Correspondingly, the amount of measurement time must be increased. It might be possible to circumvent the data-collection problem to some extent by selective excitation of only the fluorine resonances from the zinc or calcium complex, but this would create corresponding problems in performing the selective excitation.

Those skilled in the art of magnetic resonance will recognize that spin-echo methods can be used to prepare the spin systems to favor the signals of those nuclei having relatively long transverse relaxation times. The inventors have recognized a further need for improvement in the art of using a complexing agent having transverse or longitudinal relaxation times when complexed to a metal ion that differ significantly from those of the complexing agent in the free form to measure metal ion concentration using magnetic resonance imaging. Specifically, the prior art is deficient in methods of using a contrast agent to indirectly determine the concentration of a metal ion through changes in the nuclear relaxation times of a non-hydrogen imaging nucleus in the contrast agent that occur upon complexation with the metal ion. The present invention fulfills this long-standing need and desire in the art.

### SUMMARY OF THE INVENTION

The present invention is directed to a method of magnetic resonance imaging (MRI) for in vivo mapping of concentration of a target metal ion in at least one tissue. An MRI contrast agent selectively sensitive to the amount of the target metal ion is administered. The contrast agent comprises a complexing agent derivatized with a non-hydrogen imaging nucleus such that the complexing agent binds to the target metal ion. Nuclear magnetic resonance imaging signals are acquired via at least one imaging scan of the imaging nucleus and at least one image map is generated which comprises intensity of an image pixel derived from the imaging signals acquired during the imaging scan(s). Intensity of the image pixel at any point on the image map or on a subtractive composite of image maps is correlated with concentration of the target metal ion in the tissue(s) at the mapping point.

The present invention also is directed to a method of magnetic resonance imaging (MRI) for in vivo mapping of concentration of a zinc ion in at least one tissue using the method described herein. The contrast agent is 1,2-bis-(2-amino-5-fluorophenoxy)ethane-N,N,N′,N′-tetraacetic acid or 1,2-bis-(2-amino-5-trifluoromethylphenoxy)ethane-N,N,N′,N′-tetra acetic acid. The contrast agent comprises a fluorine imaging nucleus.

The present invention is directed further to methods of diagnosing a disease state and monitoring the efficacy of a therapeutic regimen to treat the disease state using the magnetic resonance imaging methods described.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 depicts the chemical structures of 5F-BAPTA and of 5T-BAPTA.

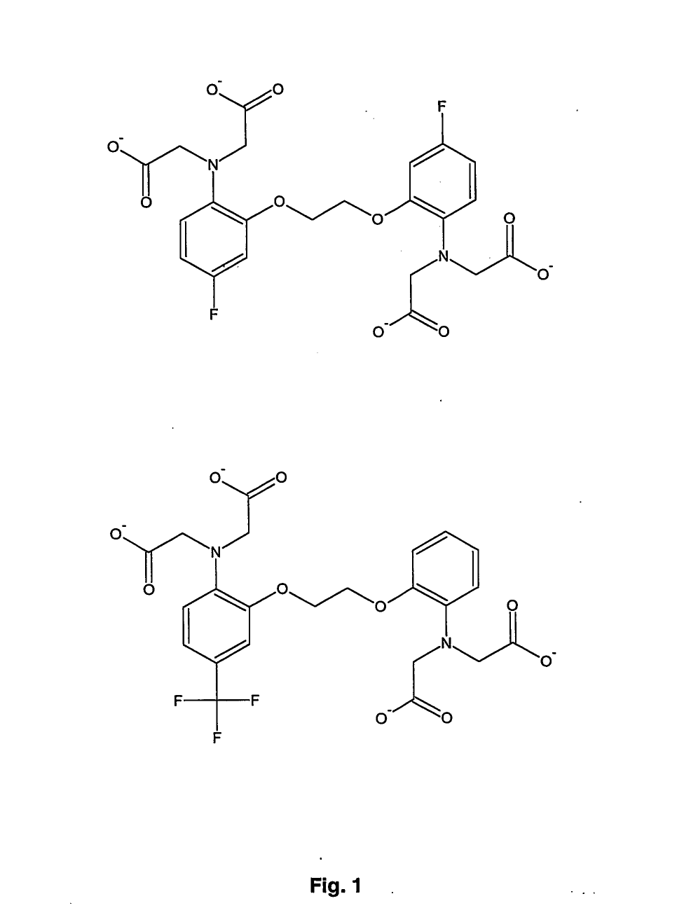

FIG. 2 depicts the fluorine transverse nuclear relaxation time T₂ for free 5T-BAPTA and 5T-BAPTA complexed with zinc.

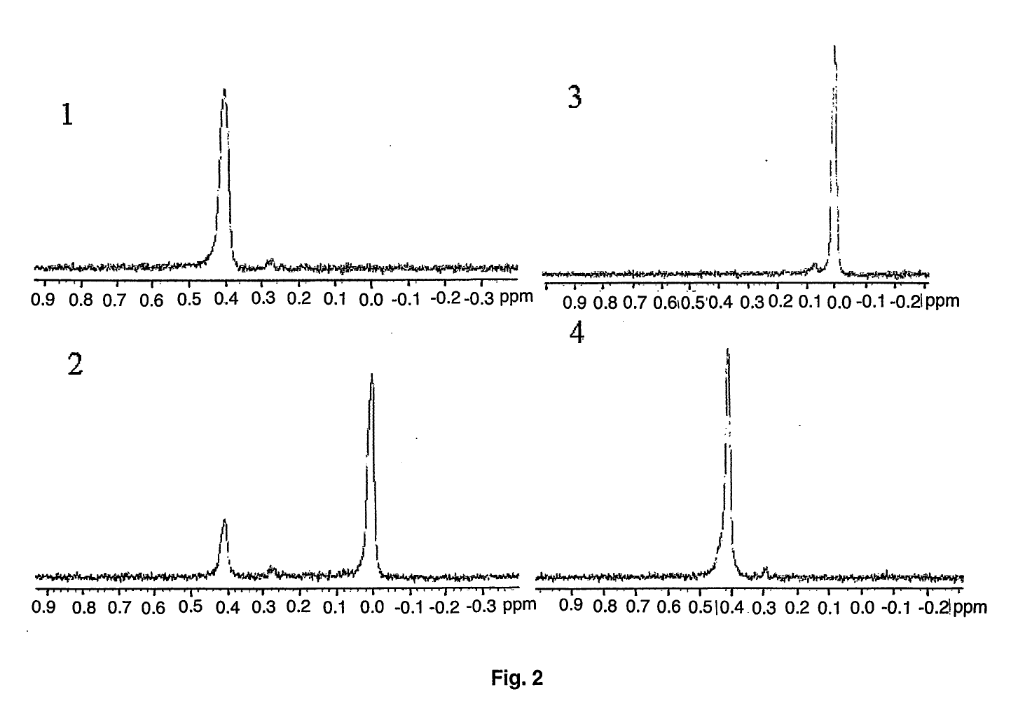

### DETAILED DESCRIPTION OF THE INVENTION

In one embodiment of the present invention there is provided a method of magnetic resonance imaging (MRI) for in vivo mapping of concentration of a target metal ion in at least one tissue, comprising the steps of administering an MRI contrast agent that is selectively sensitive to an amount of the target metal ion, where the contrast agent comprises a complexing agent having a non-hydrogen imaging nucleus and which complexing agent binds to the target metal ion; acquiring imaging signals via at least one imaging scan of the imaging nucleus; generating at least one image map comprising intensity of an image pixel derived from the imaging signals acquired during the imaging scan(s); and correlating intensity of the image pixel at any point on the image map or on a subtractive composite of the image maps with concentration of the target metal ion in the tissue(s) at the mapping point.

In all aspects of this embodiment the selectivity of the contrast agent for the target metal may be about 100-fold greater than selectivity of the contrast agent for other metal ions in vivo. Examples of the target metal ion are Zn⁺² or Cu⁺². A representative example of an imaging nucleus is ¹⁹F. The contrast agent may be administered orally, intravenously, transdermally, or via inhalation or direct administration to the tissue(s) or to an organ comprising the tissue.

In all aspects, the imaging nucleus may be introduced into the complexing agent via derivatization of the complexing agent such that one or more hydrogen atoms comprising the complexing agent are replaced by the imaging nucleus or by a functional group .comprising one or more of the imaging nuclei. Representative examples of a complexing agent are a fluorinated derivative of 1,2-bis-(2-aminophenoxy)ethane-N,N,N′,N′-tetraacetic acid, ethylene glycol bis(.beta.-aminoethyl ether)-N,N,N′,N′-tetraacetic acid or ethylenediaminetetracetic acid. Alternatively, the contrast agent may be an apo-metallothionein covalently linked with a fluorine-containing compound. An example of a fluorine-containing compound is Oregon Green.

Further to these aspects, the complexing agent may further comprise one or more functional groups which enhance in vivo biological acceptability of the contrast agent. The functional group(s) may be a targeting vector specific for a receptor. Additionally, the functional group(s) may enhance penetration of the contrasting agent across a biological barrier. An example of a biological barrier is the blood-brain barrier.

In one aspect of this embodiment binding of the target metal ion by the contrast agent measurably alters a nuclear longitudinal relaxation time of the imaging nucleus, a nuclear transverse relaxation time of the imaging nucleus or a combination thereof where intensity of the image signal from- the imaging nucleus is sensitive to the relaxation time(s). In this aspect, alteration of the longitudinal and/or transverse relaxation times independently comprises a lengthening and/or a shortening of the relaxation times of about 1.5-fold to about 15-fold of the relaxation time(s) of the contrast agent prior to binding the target metal ion. A preferred alteration of either or both of the relaxation times T₁ or T₂ is about a 2-fold to about a 5-fold shortening.

In this aspect an example of the contrast agent is 1,2-bis-(2-amino-5-fluorophenoxy)ethane-N,N,N′,N′-tetraacetic acid or 1,2-bis-(2-amino-5-trifluoromethylphenoxy)ethane-N,N,N′,N′-tetra acetic acid, the target metal ion may be Zn⁺² and the transverse relaxation time is measurably shortened. A first MRI utilizing a long delay for transverse relaxation will give an image in which low image intensity corresponds to high Zn⁺² concentration at the body site related to the image pixel. A second MRI is obtained with a short delay for transverse relaxation. Subtraction of the first image from the second yields a composite image in which high image intensity corresponds to high Zn⁺² concentration. Images sensitive to transverse relaxation are conveniently produced with a pulse sequence comprising a sequences of pulses generating a spin echo during a delay in the pulse sequence. The intensity of the spin echo will be degraded for nuclei having short transverse relaxation times.

In a related embodiment of the present invention, there is provided a method of diagnosing a disease state using the MRI methods described supra where the concentration of the target metal ion in the tissue(s) is characteristic of the presence or absence of the disease state. An example of a disease state is Alzheimer's disease and the target metal ion is Zn²⁺ or Cu²⁺. Another example of a disease state is prostate cancer and the target metal ion is Zn²⁺. Further to this embodiment the efficacy of a therapeutic regimen in treating the disease state may be monitored by using the MRI methods described supra where the concentration of the target metal ion is characteristic of the progression or regression of the disease state. The disease states and target metal ions are described supra.

In another embodiment of the present invention there is provided a method of magnetic resonance imaging (MRI) for in vivo mapping of concentration of Zn⁺² ion in at least one tissue comprising the steps of administering 1,2-bis-(2-amino-5-fluorophenoxy)ethane-N,N,N′,N′-tetraacetic acid or 1,2-bis-(2-amino-5-trifluoromethylphenoxy)ethane-N,N,N′,N′-tetra acetic acid as a contrast agent, where the fluorine imaging nucleus comprising the contrast agent is selectively sensitive to the amount of the Zn⁺² ion, acquiring imaging signals via at least one imaging scan of the imaging nucleus; generating at least one image map comprising intensity of an image pixel derived from the image signal acquired during the imaging scan(s); and correlating intensity of the image pixel at any point on the image map or on a subtractive composite of the image maps with concentration of the Zn⁺² ion in the tissue(s) at the mapping point.

In all aspects of this embodiment the functional groups further comprising the contrast agent, the effect of the functional groups on biological barriers,. the type of biological barrier and the routes of administration of the contrast agent are as described supra. Furthermore, the effect and degree of effect of binding the Zn⁺² ion on the transverse relaxation times of the fluorine imaging nucleus prior to and after binding the Zn⁺² ion are as described supra. Additionally, in a related aspect the method may comprise further steps of diagnosing and monitoring a disease state as described supra.

The following terms shall be interpreted according to the definitions set forth below. Terms not defined infra shall be interpreted according to the ordinary and standard usage in the art.

As used herein, “magnetic resonance imaging” or “MRI” shall refer to an imaging method involving detection of the nuclear magnetizations of selected nuclei in the presence of a magnetic field through the application of one or more pulses of electromagnetic radiation to the nuclei and detection of the signals generated by magnetization components transverse to the applied magnetic field generated by the pulse sequence. The abbreviation MRI is also used to refer to the images generated by such a method.

As used herein, the “applied magnetic” field shall refer to the magnetic field in which a patient or other imaging object resides during the generation of a magnetic resonance image.

As used herein, “longitudinal magnetization” shall refer to components of the total nuclear magnetization directed in the same, or in the opposite, direction as the applied magnetic field used in the generation of a magnetic resonance image. At equilibrium, the longitudinal magnetization of nuclei with a non-zero magnetic moment is non-zero in the presence of an applied magnetic field.

As used herein, “transverse magnetization” shall refer to the components of the total nuclear magnetization directed perpendicular to the direction of the applied magnetic field used in the generation of a magnetic resonance image. At equilibrium, the transverse magnetization is zero. Precession of the transverse magnetization about the applied magnetic field generates the detectable signal used in the production of a magnetic resonance image.

As used herein, “the longitudinal relaxation time” or “T₁” shall refer to the period over which the longitudinal magnetization returns to its equilibrium value after disturbance by the application of one or more pulse of electromagnetic radiation. Mathemetiacally, the longitudinal relaxation time is the time constant for exponential decay.

As used herein, “the transverse relaxation time” or “T₂” shall refer to the period over which the transverse magnetization returns to its equilibrium value of zero after disturbance by the application of one or more pulse of electromagnetic radiation. Mathematically, the longitudinal relaxation time is the time constant for exponential decay

As used herein, “target metal ion” shall refer to a metal ion whose concentration is to be detected with the invention.

As used herein, “contrast agent” shall refer to a pharmaceutical substance administered to a patient that selectively disturbs the pixel intensities in a magnetic resonance image.

As used herein, “complexing agent” shall refer to a chemical substance that binds with one or more target metal ions.

As used herein, “imaging nucleus” shall refer to the atomic nucleus that produces the measured signal used for generation of the magnetic resonance image in the practice of the invention.

As used herein, “spin echo” shall refer to a signal regenerated by one or more pulses of electromagnetic radiation after decay of the initial signal detected in a magnetic resonance experiment by destructive phase interference of different components of transverse magnetization.

The present invention provides a method of indirect measurement of the concentration of metal ions in a human or a non-human body through changes in the nuclear relaxation times. T₁ or T₂ of one or more nuclei in a molecular species induced by complexation of that species with the target metal ion. A physiologically tolerable contrast agent containing fluorine or other imaging nucleus is administered. The imaging nucleus may be detected by magnetic resonance. A sequence of radio frequency pulses suitable for generation of a magnetic resonance image is applied such that contrast in the image reflects the variations in concentration of the zinc ions in the human or non-human body. Such pulse sequences and methods of discriminating among signals of nuclei with different relaxation times are well known to those practiced in the art of magnetic resonance imaging.

The magnetic resonance signal of any nucleus other than hydrogen is suitable for the practice of the invention. The nucleus giving the signal is the imaging nucleus. Preferably, the imaging nucleus is one not normally found in high concentration in a human or non-human body. Most preferably, the imaging nucleus is ¹⁹F.

The metal ion whose concentration gradients are to be detected is the target metal ion. Target metal ions suitable for practice of the invention include, but are not limited to, Cu⁺², Ca⁺², Mg⁺², K⁺, Na⁺ or Zn⁺². A preferred targeted metal ion is Zn⁺² or Cu⁺².

The imaging nucleus comprises a complexing agent that has a high affinity for the target metal ion. Preferably, the binding species should also have a strong selective affinity for the target metal ion while having a very low affinity for other metal ions commonly found in the body. More preferably, the binding constant for complexation with the target ion will be at least 100 times that for binding to other metal ions commonly found in the body.

In the present invention binding with the target metal ion either shortens or lengthens the longitudinal T₁ and/or the transverse T₂ relaxation times of the imaging nucleus. Preferably, binding of the target metal ion with the complexing agent will shorten either or both T₁ or T₂. More preferably, binding of the target metal ion with the complexing agent will shorten either or both T₁ or T₂ of the imaging nucleus attached to the complexing agent by a factor of at least 1.5 to 15-fold. More preferably binding will shorten or lengthen either T₁ or T₂ or both by a factor of at least ten. Measurement of variations in the concentration of the target metal ion in either a human or an animal body is effected by preparation of the spin system prior to each imaging scan so as to favor the signals of nuclei having a short T₁ or those having a long T₂. Many such sequences are known to those skilled in the art of magnetic resonance imaging.

When the effect of complexation of the metal ion with the molecular species is to shorten or lengthen the longitudinal relaxation time T₁, a saturation/recovery sequence is suitable for practice of the invention. One such sequence is a series of 90° pulses separated by intervals that are short compared with the nuclear relaxation times. Thus the longitudinal magnetization is reduced to zero. Preferably the delay between pulses in the saturating pulse train should be less than one-fifth the longitudinal relaxation time.

Following the saturating pulse train, a longer delay allows for partial recovery of the longitudinal magnetization to its equilibrium value prior to imaging with one of the standard pulse procedures. The longitudinal magnetization of those nuclear species with relatively short longitudinal relaxation times will recover towards the equilibrium magnetization more fully than will those with relatively long relaxation times. Those skilled in the art of magnetic resonance will recognize that many sequences for preparing the spin system to favor the longitudinal magnetization of those nuclear species with relatively short longitudinal relaxation times are possible.

The signal to be used for creation of the image will be generated following preparation of the spin system by one of the standard methods. When the longitudinal relaxation time of the metal complex is shorter than that of the free complexing agent, the image intensity at those pixels corresponding to spatial regions rich in the target metal ion will be more intense than those pixels corresponding to spatial regions that are weak in the target metal ion.

When the effect of complexation of the metal ion with the molecular species is to shorten or lengthen the transverse relaxation time T₂, a spin-echo sequence is suitable. The simplest sequence consists of an exciting pulse, which might be, but is not limited to, a 90° pulse, followed after a delay that is short compared with the transverse relaxation times of the nuclei by an inverting pulse. In general, the pulse delay will be equal to or less than the transverse relaxation time. The signal is then observed as a spin echo following a second delay equal to the first. Spin-echo sequences are routinely used for the generation of magnetic resonance images. Those skilled in the art will recognize that a train of refocusing pulses can also be used to generate a spin echo.

The observed magnetic resonance signal is processed with standard techniques to generate a magnetic resonance image in which contrast will reflect the variation in the concentration of the target metal ion throughout the imaged portion of the animate human or non-human body. When the effect of complexation of the complexing agent with the target metal ion is to shorten the transverse relaxation time of the imaging nucleus, the intensity of those pixels corresponding to those spatial regions is less than that of pixels corresponding to spatial regions that are weak in the target metal ion.

The intensity difference is greatest when the pulse sequence has a relatively long delay for generation of the spin echo. Thus, an image is created in which high image intensity corresponds to high concentrations of the target metal ion by subtraction of an image acquired with a long delay from one acquired with a short delay. That is image intensity in the first image will be relatively low and will be skewed in favor of those pixels corresponding to spatial regions in which the zinc concentration is low. The overall intensity in the second image will be higher and will be relatively insensitive to the zinc concentration. Prior to the subtraction, the image acquired is multiplied with a long delay by a scaling factor.

Generally, about a 2 to 5% signal variation is sufficient to generate contrast in a magnetic resonance image. Thus, it is preferred that target metal ions of the invention modify the nuclear relaxation times sufficiently that the observed signals increase or decrease by about 2% to about 5%. An increase or decrease of about 2% to about 10% is more preferred and of about 10% to about 50% is most preferred.

The practice of the invention requires the administration to an animate human or non-human body of a suitable derivative of a complexing agent, the derivitization of which involves introduction of the non-hydrogen imaging nucleus. Such derivitization may include, but is not limited to, replacement of one or more hydrogen atoms in the complexing agents with fluorines. Alternatively, it may involve replacement of one or more hydrogen atoms with trifluoromethyl groups. It will be obvious to those practiced in the art of chemical modification that many other suitable types of derivitization for the introduction of fluorine atoms are possible. It is also obvious that there are suitable methods for the introduction of other imaging nuclei, such as phosphorous, carbon-13, or nitrogen-15.

When Zn⁺² or Ca⁺² is the target metal ion, suitable complexing agents include, but are not limited to, fluorinated derivatives of 1,2-bis-(2-amino-phenoxy)ethane-N,N,N′,N′-tetraacetic acid (BAPTA), ethylene glycol bis(.beta.-aminoethyl ether)-N,N,N′,N′-tetraacetic acid (EGTA); ethylenediaminetetracetic acid (EDTA). It is contemplated that 5F-BAPTA and 5T-BAPTA are suitable fluorinated derivatives. For example, 5F-BAPTA and 5T-BAPTA exhibit a large reduction in the fluorine transverse relaxation time, respectively, upon complexation with Zn⁺². Other suitable species for the practice of the invention are possible. In a preferred embodiment, the chelators of the invention include one or more substituent groups that serve as functional groups for chemical attachment or as solubilizing groups. Suitable functional groups include, but are not limited to, amino, preferably primary amino, carboxylate, phosphate, hydroxy, oxyacid, thiol groups and C₁₋₆ alkyl groups substituted with one or more such groups.

It is further contemplated that metallothioneins may be used as complexing agents. Metallothioneins (MTs) are a family of four small, cysteine-rich proteins that bind to heavy metals, particularly zinc ions, in the body. In adult mice, MT-1 and MT-2 are found in all organs, MT-3 is expressed mostly in brain (17) and MT-4 is most abundant in epithelial tissue.

Each MT has about 60 amino acids, including 18-20 cysteine residues (18). The sulfhydryl groups of the cysteine residues bind diverse metal ions, such as Zn²⁺ and Cd²⁺ in multiple metal-thiolate clusters (19) to form a holo-MT. The metal ion binding by an apo-MT induces folding of the metallothionein back bone into a specific three-dimensional structure (20). Reaction between metal ions and apoMT has been shown to occur within milliseconds (20).

In a holo-MT the metal ions hold the backbone of the protein rigidly in place. The reorientational motion necessary to nuclear relaxation is highly restricted. Removal of the metal ions, thereby forming an apo-MT, greatly facilitates local reorientational motions of the chains.

Methods of removing zinc ions from holo-MTs to form apo-MTs are known in the art. In general one method uses a Sephadex G-15 column equilibrated with 0.01N HCl to separate the Zn²⁺ ions from the holo-MT. Completeness of dissociation of Zn²⁺ from isolated apo-MT is checked by atomic absorption spectrophotometry. The concentration of apo-MT is measured by assaying thiol groups with DTNB (20).

Alternatively, a method used to prepare an apo-carbonic anhydrase II may be used (21). Metal ions are removed by using Amicon diaflow filtration against 50 mM dipicolinic acid (DPA) at pH 7.0, followed by gel firltration on Sephadex G-19 to remove dipicolinate. Buffers used are stripped metal impurities by passage over Chelex-100 columns.

Oregon Green is a derivative of fluorescein containing two fluorine atoms. It is commercially available in various reactive forms. The isocyanate derivative is well known to be reactive towards amino group and is widely used for labeling proteins. Attachment of Oregon Green to an apo-metallothionein yields a composition giving a fluorine NMR signal and thus functions as a contrast agent comprising a non-hydrogen imaging nucleus. Local motion of the protein backbone and the attached dye molecule will be sufficiently fast that the rotational correlation time will be short in comparison to the inverse of the Larmor frequency of the fluorine nuclei. The longitudinal and transverse relaxation times T₁ and T₂ of the fluorine will be relatively long.

Complexation of the substituted metallothionein with zinc inhibits the local motion of the protein chains and the attached dye molecule. The rotational correlation time is lengthened. When the rotational motion is sufficiently fast that the rotational correlation time is short compared with the inverse of the Larmor frequency, lengthening the correlation time shortens both the longitudinal relaxation time T₁ and the transverse relaxation time T₂.

BAPTA and many derivatives thereof have a doubly negative charge. These are preferentially soluble in water instead of organic liquids, such as octanol, and lipids in the body. Complexation with Zn⁺² or another dipositive metal ion leads to a neutral species that has an elevated solubility in organic solvents. When the metal complex is dissolved in an organic solvent, the relaxation times of the nuclei in the complexing agent are different from those of the metal complex dissolved in water. Also, they are different from those of the free complexing agent dissolved in water. The change in the relaxation times of the complexing agent when it binds with the metal ion and it is transferred to the organic phase makes possible the generation of a magnetic resonance image sensitive to the zinc concentration in the body through the use of pulse sequences that weight the intensity of the image in terms of the relaxation times of the observed nucleus.

It may be necessary to tailor the solubility of the complexing agent, with and without a bound metal, through attachment of appropriate substituents to the complexing agent. Substituents that enhance the solubility in organic solvents include alkyl groups and other substituents derived from water-insoluble molecules. For some complexing agents, binding with a metal ion significantly changes the relaxation times without a need for transfer of the metal complex to an organic phase. For these materials it is not necessary to enhance the solubility of the agent in an organic phase. Substituents that enhance the water solubility include ionizable groups such as carboxylic acid groups and substituents that form strong hydrogen bonds with water such as hydroxyl and amino groups.

A preferred embodiment utilizes complexes that cross the blood-brain barrier. For some applications of the instant invention such as monitoring the accumulation of zinc in the brain in connection with the development of Alzheimer's disease, it may be desirable that the free complexing agent readily passes across the blood-brain barrier. Enhancement of the solubility of the complexing agent in organic solvents has the beneficial effect of facilitating transfer of the agent across the blood-brain barrier. For example, as is known in the art, a DOTA derivative with one of the carboxylic acids replaced by an alcohol to form a neutral DOTA derivative has been shown to cross the blood-brain barrier. Similar modifications will be suitable to allow the complexing agents of the present invention to cross the blood-brain barrier.

Examples of substituent groups that will facilitate passage of the complexing agents through the blood/brain barrier are groups that enhance the solubility of the agent in lipid phases of the body. These include long-chain alky groups and other lipophilic substituents. Charged substituent groups, in which the charge of the group cancel other charged substituents of the complexing agent may also be useful. For example, the positive charge on an ammonium group will effectively cancel the negative charge on a carboxylate group at another location on the molecule. Internal cancellation of the molecular charge of the complexing agent will decrease the water solubility and enhance the lipid solubility of the agent, thus facilitating passage of the agent through the blood/brain barrier.

It is further anticipated that the agent may contain substituents to facilitate passage of the agent through the blood/brain barrier, but that the agents are attached to the complexing agent by a linking group that is subject to attack by enzymes in the organ to be imaged which may be the brain. Thus, as a result of the presence of subsitutent groups, the agent can be water insoluble as administered and will pass readily through the blood/brain barrier or other biological barrier. In the organ to be imaged, the substituent groups are cleaved and the agent will become water soluble. As a water-soluble substance, the agent will have greater affinity for the target metal ions than as a water-insoluble substance.

To provide selectivity, the complexing agent may be targeted passively or actively to regions of diagnostic interest, such as organs, vessels, or sites of disease. Thus, the complexing agents of the present invention may comprise one or more targeting groups to allow them to accumulate in or to be selectively retained by or to be slowly eliminated from certain parts of the body, such as specific organs, parts of organs, bodily structures and disease structures and lesions. These may be attached directly to the body of the complexing agent or through one of the functional groups described supra.

The targeting groups may operate by either an active or a passive mechanism. Active targeting involves modification of the biodistribution of the complexing agent because there is attachment to the complexing agent through a targeting vector to one or more receptor species present in the tissue of the organism to be imaged. Such binding will effectively decrease the rate of loss of the complexing agent from the tissue of interest. Appropriate targeting vectors include, but are not limited to, amino acids, peptides, antigens, haptens, enzyme substrates, enzyme cofactors, enzyme inhibitors, biotin, hormones, neurohormones, neurotransmitters, growth factors, lymphokines, lectins, toxins, carbohydrates, oligosaccharides, polysaccharides, dextrans, oligonucleotides stabilized against nucleases, receptor-binding drugs and ligands, antibodies, and functional fragments thereof.

Optionally, the complexing agent can be distributed to the tissue of interest by a passive mechanism not involving specific interaction of the complexing agent with a chemical site in the tissue. In this case, the substituents on the complexing agent will act to change a physical property such as the solubility or diffusivity of the agent. Optionally, the complexing agent may be enclosed in micelles or liposomes. Suitable solubilizing groups include hydroxyl, 1,2-dihydroxyethyl, 1,2-dihydroxypropyloxy, carboxyl, sulfonate, phosphonate, and poly(alkylene oxidyl) groups such as hydroxypoly(ethylene oxidyl) and methoxypoly(ethylene oxidyl), the weights of which can be up to about 50,000.

Pharmaceutical compositions comprising pharmaceutically acceptable salts of the contrast agents can also be prepared by using a base to neutralize the complexes while they are still in solution. Some complexing agents that are suitable for the practice of the invention will be formally uncharged and will not need counterions. In some embodiments, it may be desirable to increase the blood clearance times or half-life of the contrast agents of the invention. For example, U.S. Pat. No. 5,155,215 discloses adding carbohydrate polymers to the chelator. Thus, one embodiment utilizes polysaccharides as substitution groups on the complexing agents.

The medium to be administered in the practice of the invention will contain the contrast agent comprising the complexing agent derivative and conventional pharmaceutical formulation aids such as wetting agents, buffers, disintegrants, binders, fillers, flavoring agents, and a liquid carrier medium such as sterile water, water/ethanol mixtures, and so forth as are known in the art. The formulated system should be suitable for injection, inhalation, catheterization or transdermal introduction. For oral administration, the pH of the composition is preferably from about pH 2 to about pH 7; buffers or adjusting agents may be used if necessary to bring the pH into this range. Additionally, dosage and doses, as well as schedules of administration, are well known within the art.

Preferably, the contrast agent will distribute almost uniformly in the organ to be imaged. When it does, contrast will be determined entirely by the different extent to which the contrast agent is complexed to zinc ions, or other metal ions, in different regions of the organ. However, the invention will also be useful in cases in which the agent is distributed unequally within the organ.

It is also contemplated that the magnetic resonance imaging methods described herein can be used to diagnose a disease state. The disease state may be characterized by a concentration or lack thereof of the target metal ion in the tissue, tissues or organs affected by the disease. Comparing the concentration of the target metal ion determined by the image signal map with those characteristic concentrations of the metal ion indicates the presence or absence of the disease. Levels of Zn⁺² or Cu⁺² can be used as a diagnostic indicator for Alzheimer's disease. Zn⁺² can also be used to detect prostate cancer. Furthermore, the present invention can be used to monitor a course of therapeutic treatment for a disease state. The concentration levels of the target metal ions in the tissue, tissues or organ exhibiting the diseased state can be indicates the severity, i.e., the progression or regression, of the disease state.

The present invention offers a number of advantages in the mapping of metal ion concentrations. A contrast agent containing a non-hydrogen imaging nucleus is provided. Utilization of a non-hydrogen imaging nucleus such as, but not limited to, fluorine that is extremely rare in the body means that the background signals in the measurement will be low. Furthermore, fluorine is readily introduced into many organic molecules as a substituent. This provides significant freedom for the design of contrast agents containing fluorine using known synthetic methods.

As described herein, the invention provides a number of therapeutic advantages and uses. The embodiments and variations described in detail herein are to be interpreted by the appended claims and equivalents thereof. The following examples are given for the purpose of illustrating various embodiments of the invention and are not meant to limit the present invention in any fashion.

### EXAMPLE 1

NMR Data Acquisition

All fluorine NMR spectra were collected with a 400 MHz Oxford/Varian Unity Plus spectrometer at 25° C. The nominal 90° pulse width was 7.8 μsec. Transverse relaxation times were determined by the best fit of data collected using the Carr-Purcell-Meiboom-Gill sequence supplied with the spectrometer.

### EXAMPLE 2

Nuclear Relaxation Times T1 and T2 of 5T-BAPTA

A 1 M solution of Ultra Pure ZnCl₂ (Sigma) was prepared and diluted in a serial fashion to produce intermediate stock solutions of concentration 100 mM and 10 mM in water. A 200 mM solution of 5-T-BAPTA (Molecular Probes) was prepared and added to aliquots of either zinc solution, as appropriate, in sufficient quantity to yield a final, fixed concentration of 2 mM of the complexing agent. Each sample had a final volume of 750 μL and contained 10% D₂O to provide a lock signal.

The transverse relaxation time T₂ of the complex measured for Sample 1 appears to be only ⅕ as large as that of the free complexing agent measured from Sample 3. Furthermore, the relaxation time of the bound species in Sample 2 is shorter than that of the free species in the same solution. Table 1 shows the T₂ times for complexed and free 5T-BAPTA.

Experimental artifacts could affect the apparent magnitude of the relaxation times. Furthermore, the relaxation times at the relatively low magnetic field strengths typically used for medical imaging are likely to differ somewhat from those measured at high magnetic field strengths. When viewed as a whole, the results suggest that there is a real difference in the relaxation times of the bound and free species that is exploitable for creation of a zinc-sensitive magnetic resonance map.

TABLE 1
Transverse relaxation times for 5T-BAPTA bound to zinc and free

| | ZnCl₂ | MgCl₂ | CaCl₂ | T₂ bound | T₂ free |
|---|---|---|---|---|---|
| Sample | (mM) | (mM) | (mM) | (sec) | (sec) |
| 1 | 2.0 | 1.0 | 2.0 | 0.14 ± 0.03 | |
| 2 | 0.5 | 1.5 | | 0.265 ± 0.025 | 0.387 ± 0.012 |
| 3 | 0 | 1.65 | 3.35 | | 0.763 ± 0.032 |
| 4 | 3.5 | 0.5 | 1.0 | 0.216 ± 0.007 | |

### EXAMPLE 3

Nuclear Relaxation Times T1 and T2 of 5F-BAPTA

A solution of 0.00315 g (4.738 micromoles) 5F-BAPTA and 0.013 g (54.9 micromoles) HEPES buffer in 0.5 ml deionized water and 0.2 ml deuterium oxide was prepared. To this was added 47.4 microliters (4.738 micromoles) 0.1 M zinc sulfate solution. An equivalent volume (0.7 ml) n-octanol was added, and the mixture was shaken vigorously. The organic and aqueous phases were separated by pipette and placed in NMR tubes. To the octanol solution was added 0.1 ml deuterated methanol to provide an NMR lock signal. A reference sample without zinc was prepared in a similar fashion except that there was no octanol extraction.

The aqueous solution without zinc gave a single signal that had barely resolvable fine structure from proton-fluorine coupling. The T₁ value was 0.82±0.01 sec and the T₂ value was 0.080±0.003 sec. Both the aqueous and the octanol solutions with zinc gave only a single NMR peak. The aqueous peak was about 3.8 ppm to higher frequency of that in the solution without zinc. The signal-to-noise of the aqueous solution was 33 times that of the of the octanol solution, indicating that the zinc complex is still highly water soluble.

The T₁ value of the octanol solution was 0.53±0.01 sec. Complexation of the 5F-BAPTA with Zn⁺⁺ leads to some octanol solubility. In octanol, the zinc complex has a significantly shorter longitudinal relaxation time than does the free complexing agent in water. The T₂ value of the zinc complex in octanol is 0.013±0.001 sec. Thus, there is a strong reduction in the transverse relaxation time upon complexation of the 5F-BAPTA with zinc and transfer into the organic solvent.

The T₁ value of the aqueous solution of the zinc complex was 0.82±0.01 sec. There is no significant effect of complexation of the 5F-BAPTA with Zn⁺⁺ in water. The T₂ value of the aqueous solution of the zinc complex was 0.0248±0.0003. There is a significant reduction in the value of the transverse time. As such, spin-echo sequences to generate magnetic resonance images that are sensitive to zinc concentration can be constructed.

The following references are cited herein:
1. Saaranen, M. et al., Hum. Reprod., 2, pp. 475 (1987)
2. Canale, D. et al., Int. J. Androl., 9, pp. 477 (1986)
3. Chia, S. E. et al., J. Androl., 21, pp. 53 (2000)
4. Lovell, M. A. et al., J. Neurol. Sci., 158, pp. 47 (1998)
5. Willoughby et al., Exp. Neurol., 118(3), pp. 332-9 (1992)
6. Gentlemann et al., Prog. Brain Res., 96, pp. 237-46 (1993)
7. Stephenson et al., Brain Res., 593, pp. 128 (1992)
8. Ahn, et al., Exp. Nerl., 154(1), pp. 47-56 (1998)
9. Koh et al., Science, 272, pp. 1013-1016 (1996)
10. Lees, et al, Brain Res., 799, pp. 108-117 (1998)
11. Suh et al., Soc. for Neuroscience Abstr., 21, pp. 216 (1995)
12. Suh et al., Soc. for Neuroscience Abstr., 22, pp. 2101 (1996)
13. Suh et al., J. Histochem. Cytochem., 47, pp. 969-72 (1999)
14. Benters, J. et al., Biochem. J., 322, pp. 793 (1997)
15. Song, S. K. et al., Am. J. Physiol., 269, C318 (1995)
16. Hanaoka, K. et al., J. Chem. Soc., , 2, pp. 1840-43 (2001)
17. Palmiter et al., Proc Nat Acad Sci, 89, pp. 6333-6337 (1992)
18. Hamer et al., Annu Rev Biochem, 55, pp. 913-951 (1986)
19. Narula et al., Biochemistry, 34, pp. 6620-631 (1995)
20. Ejnik et al., J Inorg biochem, 88, pp. 144-152 (2002)
21. Alexander et al., Biochemistry, 32, pp. 1510-1518 (1993)

Any patents or publications mentioned in this specification are indicative of the levels of those skilled in the art to which the invention pertains. Further, these patents and publications are incorporated by reference herein to the same extent as if each individual publication was specifically and individually incorporated by reference.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A method of magnetic resonance imaging (MRI) for in vivo mapping of concentration of a target metal ion in at least one tissue, comprising the steps of:
administering an MRI contrast agent, said contrast agent selectively sensitive to amount of said target metal ion, said contrast agent comprising:
a complexing agent comprising a non-hydrogen imaging nucleus, said complexing agent binding to said target metal ion;
acquiring imaging signals via at least one imaging scan of said imaging nucleus;
generating at least one image map comprising intensity of an image pixel derived from said imaging signal acquired during said imaging scan(s); and
correlating intensity of said image pixel at any point on said image map or on a subtractive composite of said image maps with concentration of said target metal ion in the tissue(s) at said mapping point.

2. The method of claim 1, wherein selectivity of the contrast agent for said target metal ion is about 100-fold greater than selectivity of the contrast agent for other metal ions in vivo.

3. The method of claim 1, wherein the target metal ion is Zn⁺² or Cu⁺².

4. The method of claim 1, wherein the imaging nucleus is ¹⁹F.

5. The method of claim 1, wherein said imaging nucleus is introduced into said complexing agent via derivatization of said complexing agent such that one or more hydrogen atoms comprising said complexing agent are replaced by said imaging nucleus or by a functional group comprising one or more of said imaging nuclei.

6. The method of claim 5, wherein the complexing agent is a fluorinated derivative of 1,2-bis-(2-aminophenoxy)ethane-N,N,N′,N′-tetraacetic acid, ethylene glycol bis(.beta.-aminoethyl ether)-N,N,N′,N′-tetraacetic acid or ethylenediaminetetracetic acid.

7. The method of claim 1, wherein the complexing agent is an apo-metallothionein covalently linked with a fluorine-containing compound.

8. The method of claim 7, wherein the fluorine-containing compound is Oregon Green.

9. The method of claim 1, said complexing agent further comprising one or more functional groups, said functional groups enhancing in vivo biological acceptability of the contrast agent.

10. The method of claim 9, wherein the functional group(s) comprises a targeting vector specific for a receptor.

11. The method of claim 8, wherein the functional group(s) enhances penetration of the contrasting agent across a biological barrier.

12. The method of claim 10, wherein the biological barrier is the blood-brain barrier.

13. The method of claim 1, wherein the contrast agent is administered orally, intravenously, transdermally, or via inhalation or direct administration to the tissue(s) or to an organ comprising the tissue.

14. The method of claim 1, wherein binding said target metal ion by the contrast agent measurably alters a nuclear longitudinal relaxation time of said imaging nucleus, a nuclear transverse relaxation time of said imaging nucleus or a combination thereof, wherein intensity of said image signal from said imaging nucleus is sensitive to said relaxation time(s).

15. The method of claim 14, wherein alteration of said longitudinal and/or transverse relaxation times independently comprises a lengthening and/or a shortening of said relaxation times of about 1.5-fold to about 15-fold of the relaxation time(s) of the contrast agent prior to binding said target metal ion.

16. The method of claim 15, wherein said longitudinal and said transverse relaxation times are shortened about 2-fold to about 7-fold.

17. The method of claim 14, wherein the contrast agent is 1,2-bis-(2-amino-5-fluorophenoxy)ethane-N,N,N′,N′-tetraacetic acid or 1,2-bis-(2-amino-5-trifluoromethylphenoxy)ethane-N,N,N′,N′-tetra acetic acid, said target metal ion is Zn⁺² and said transverse relaxation time is measurably shortened.

18. The method of claim 17, wherein a first MRI image map utilizing a long relaxation delay for transverse relaxation (T₂) and a second MRI image map utilizing a short relaxation delay for transverse relaxation are generated from imaging scans such that said first MRI image map is subtracted from said second MRI image map thereby obtaining a high intensity image signal map of said Zn⁺² concentrations.

19. The method of claim 18, wherein the imaging scan utilizes a spin-echo sequence.

20. The method of claim 1, further comprising the step of:
diagnosing a disease state wherein the concentration of said target metal ion in the tissue(s) is characteristic of the presence or absence of the disease state.

21. The method of claim 20, wherein the disease state is Alzheimer's disease and the target metal ion is Zn⁺² or Cu⁺².

22. The method of claim 20, wherein the disease state is prostate cancer and the target metal ion is Zn⁺².

23. The method of claim 20, further comprising the step of:
monitoring the efficacy of a therapeutic regimen to treat said disease state wherein the concentration of said target metal ion in the tissue(s) is characteristic of progression or regression of the disease state.

24. A method of magnetic resonance imaging (MRI) for in vivo mapping of concentration of Zn⁺² ion in at least one tissue, comprising the steps of:
administering 1,2-bis-(2-amino-5-fluorophenoxy)ethane-N,N,N′,N′-tetraacetic acid or 1,2-bis-(2-amino-5-trifluoromethylphenoxy)ethane-N,N,N′,N′-tetra acetic acid as a contrast agent, wherein the fluorine imaging nucleus comprising the contrast agent is selectively sensitive to the amount of the Zn⁺² ion,
acquiring imaging signals via at least one imaging scan of said imaging nucleus;
generating at least one image map comprising intensity of an image pixel derived from said image signal acquired during said imaging scan(s); and
correlating intensity of said image pixel at any point o n said image map or on a subtractive composite of said image maps with concentration of the Zn⁺² ion in the tissue(s) at said mapping point.

25. The method of claim 24, said contrast agent further comprising one or more functional groups, said functional groups enhancing in vivo biological acceptability of the contrast agent.

26. The method of claim 25, wherein the functional group(s) comprises a targeting vector specific for a receptor.

27. The method of claim 25, wherein the functional group(s) enhances penetration of the contrasting agent across a biological barrier.

28. The method of claim 27, wherein the biological barrier is the blood-brain barrier.

29. The method of claim 24, wherein the contrast agent is administered orally, intravenously, transdermally, or via inhalation or direct administration to the tissue(s), or to an organ comprising the tissue.

30. The method of claim 1, wherein binding the Zn⁺² ion by the contrast agent measurably shortens a nuclear transverse relaxation time of said fluorine imaging nucleus, wherein intensity of said image signal from said imaging nucleus is sensitive to said transverse relaxation time.

31. The method of claim 30, wherein said transverse relaxation time is shortened about 1.5-fold to about 15-fold of the transverse relaxation time of the contrast agent prior to binding the Zn⁺² ion.

32. The method of claim 30, wherein said transverse relaxation time is shortened about 2-fold to about 7-fold.

33. The method of claim 24, wherein the imaging scan utilizes a spin-echo sequence.

34. The method of claim 24, further comprising the step of:
diagnosing a disease state wherein the concentration of said target metal ion in the tissue(s) is characteristic of the presence or absence of the disease state.

35. The method of claim 34, wherein the disease state is Alzheimer's disease or prostate cancer.

36. The method of claim 34, further comprising the step of:
monitoring the efficacy of a therapeutic regimen to treat said disease state wherein the concentration of the Zn⁺² ion in the tissue(s) is characteristic of progression or regression of the disease state.
